(19) United States
(12) Patent Application Publication
(10) Pub. No.: US 2017/0100067 A1
(43) Pub. Date: Apr. 13, 2017

(54) Method and system for emotion measurement

(71) Applicant: Yadong Liu, (US)

(72) Inventor: Yadong Liu, San Clemente, CA (US)

(21) Appl. No.: 14/881,139

(22) Filed: Oct. 12, 2015

(51) Int. Cl. A61B 5/16; A61B 7/02; A61B 5/00

(52) U.S. Cl. CPC A61B 5/165; A61B 5/6812; A61B 5/0004; A61B 5/742; A61B 5/6803; A61B 5/7282; A61B 7/02

(57) ABSTRACT

Techniques for measuring the emotional state of minds in a person based upon a set of biological data captured from the person in a natural environment, where the person is not restricted at all. Various signals from sensors are captured with or without the intervention of the person. These signals are processed to be converted to sensor data. A dedicated computing device working as a server is provided to collect the sensor data along with other necessary data available on the Internet, where the measurement of the emotion is measured, derived or calculated based on the sensor data, the fetched necessary data and/or the historical measurements.

### BACKGROUND OF THE INVENTION

Field of the Invention

The present invention is generally related to the area of data communication between a client and a server over the Internet. Particularly, the present invention is related to techniques for evaluating, measuring or determining an emotional state of minds in humans (a.k.a., emotion).

Description of the Related Art

Detecting emotional information begins with passive sensors which capture data as an input about a physical state or behavior of a human being without interpreting the input. The data gathered is analogous to the cues humans use to perceive emotions in others. For example, a video camera might capture facial expressions, body posture and gestures, while a microphone might capture speech. Other sensors detect emotional cues by directly measuring physiology data, such as skin temperature, galvanic resistance, pulses and etc.

Recognizing emotional information requires the extraction of meaningful patterns from the gathered data. This is done using machine learning techniques that process different modalities, such as speech recognition, natural language processing or facial expression detection, and produce either labels (i.e., confused) or coordinates in a valence-arousal space. From business perspective, studies have shown that there are enormous needs to measure the emotional state of minds in humans for market research, educational, and medical purposes.

In the past, researchers would have to attach different types of sensors to a human body in order to capture the vital signals. A participant for such measurement is essentially limited to a confined space with little freedom to move around. Such measurements are generally considered artificial or limited and not accurate in a sense that the participant has already been set up in an environment he or she is not used to. There is a great need for measuring the emotional state of minds in a natural environment in which a participant often lives or how a participant reacts to events that may have happened expectedly or unexpectedly.

It is commonly known that an emotional state of minds (i.e., emotion) is not purely dictated at the moment the emotion is measured. In other words, an instantaneous emotion measurement is not much useful but could potentially lead to a wrong judgment. The emotion of a human being, even though changing from time to time, is intertwined psychologically and physically with many surrounding elements (e.g., weather, temperature, sudden event and etc). Thus there is another need that the measurement of the emotional state of minds in a person is conducted in connection with other information that may be related to the person, his/her location and vicinity and circumstance he/she may be in or related to.

Many existing emotional measurements on human beings are isolated in a sense that the results are viewed alone. There could be occasions that a majority of people in a particular region may have their own emotional measurements increased or decreased due to certain events or conditions in the region. An isolated view of an measurement may lead to or cause an unnecessary alarm. Thus there is another need for mechanisms that provide possible comparisons of a result with others at the time of concluding a measurement.

The current emotional measurements require a set of special sensors attached to a person. With a set of dedicated equipments, the sensor data is read out and comprehended by one or more trained professionals. Thus there is still another need for average persons to get their emotional measurements without much training and with commercially available wearable devices.

### SUMMARY OF THE INVENTION

This section is for the purpose of summarizing some aspects of the present invention and to briefly introduce some preferred embodiments. Simplifications or omissions may be made to avoid obscuring the purpose of the section. Such simplifications or omissions are not intended to limit the scope of the present invention.

In general, the present invention is related to measuring the emotional state of minds in a person based upon a set of biological data captured from the person. One of the advantages, objects and benefits in the present invention is that the emotional state of minds (a.k.a., emotion) of the person is measured, derived or calculated in a natural environment. There is almost no restriction on the person. Various signals from sensors are captured with or without the intervention of the person. These signals are processed (e.g., via analog to digital convention or ADC) to be converted to sensor data. A dedicated computing device working as a server is provided to collect the sensor data along with other necessary data available on the Internet, where the measurement of the emotion is measured, derived or calculated based on the sensor data, the fetched necessary data and/or the historical measurements.

According to one aspect of the present invention, the biological data is from sensor signals largely captured by a plurality of sensors enclosed in one or more wearable devices. With a mobile device (e.g., a smartphone), the collected biological data is transported to a designated server device that is caused to execute a server module specifically invented, uniquely designed, implemented or configured to derive the emotion of the person at the time of some of the biological data are captured.

To account for possible external events and conditions that may have a significant impact on the person, various external data sources are incorporated to derive the emotion. According to another aspect of the present invention, a set of predefined network resources are accessed to obtain data that may have some impact on the emotion of the person before, during or right after the biological data is captured from the person.

According to still another aspect of the present invention, commercially available wearable devices are utilized. As some of them are worn on different parts of the body (e.g., Apple Watch on a wrist while Google Glass on a head), biological data from different parts of the body is captured and collectively utilized in determining the emotion of the person.

To facilitate the expression of the emotion in a form understood to the general public, the derived emotion is expressed in an index or numerals with a range according to another aspect of the invention. Logically, the two extremes on the two opposite ends of the range represent respectively the worst or best mind mode or feeling that could ever happen to a normal person. Such an expression can be not only understood to the general public but also used to induce or call for a specific service or a message (e.g., an advertisement).

According to another aspect of the invention, a derived emotion measurement is compared with historical measurements of the person and/or with that of others in the vicinity of the person. A comprehended measurement is concluded before the derived emotion measurement is delivered to the person, for example, to avoid unnecessary alarming or to present a more realistic result.

According to yet another aspect of the invention, additional services or goods in connection with the measured emotion are provided in connection with the measured emotion in the range.

The present invention may be implemented in software or in a combination of software and hardware, and practiced as a system, a process, or a method. According to one embodiment, the present invention is a method for measuring an emotion, the method comprises: retrieving a profile of a user; sending a request by a server device to a client device to capture some or all of predefined biological data from the user, wherein at least a part of the client device is wearable and includes a plurality of sensors generating different sensing data; receiving the biological data from the client device; feeding the biological data to a data processing unit together with other data; providing processed data to an emotion measurement engine configured to derive the emotion from the processed data; and causing the client device to display the derived emotion to the user.

According to another embodiment, the present invention is a mobile device for measuring an emotion, the mobile device being carried by a user comprises: a plurality of sensors; a processor; a wireless interface to allow the mobile device to communicate with a server device wirelessly over a data network; a memory space, coupled to the process, provided to store a client module, wherein the client module is executed by the process to cause the mobile device to perform operations of:
collecting some or all of predefined biological data of the user from the sensors in response to a request from the server device to capture the biological data;
transporting the biological data to the server device;
receiving a derived emotion measurement from the server device, wherein the server device is configured to derive the emotion of the user from the biological data and other data; and
displaying the derived emotion to the user.

One of the objects, features, and advantages of the present invention is to measure the emotion of a person by using the commercially available wearable devices and present the emotion measurement whenever or wherever the person needs.

Other objects, features, and advantages of the present invention will become apparent upon examining the following detailed description of an embodiment thereof, taken in conjunction with the attached drawings.

### BRIEF DESCRIPTION OF THE DRAWINGS

These and other features, aspects, and advantages of the present invention will become better understood with regard to the following description, appended claims, and accompanying drawings where:

FIG. 1A shows a basic system configuration in which the present invention may be practiced in accordance with one embodiment thereof;

FIG. 1B shows some of the commercially available wearable devices that may be used to collect one or more types of the biological data;

FIG. 1C illustrates an internal functional block diagram of an exemplary wearable device or a client device that may be used as a client in FIG. 1A;

FIG. 2A shows a logic relationship between a client and a server, where the client represents one of many clients that are intended to communicate with the server;

FIG. 2B shows two wearable devices, a watch (e.g., Apple Watch) and a pair of glasses (e.g., Google glass) that may be used to capture some of the biological data;

FIG. 2C illustrates that the biological data captured from a user is transported to a server with other external data;

FIG. 3A and FIG. 3B collectively show a flowchart or process of determining an emotion of a user from the biological data captured directly from the user and other available data from the Internet;

FIG. 3C shows an example of a display to show a numerical expression of the measured emotion;

FIG. 4A shows a functional block diagram of a server in which a server module resides in a memory space and is executed by one or more processors;

FIG. 4B shows a functional block diagram; and

FIG. 4C shows a diagram of comparing the measurement in FIG. 4B with others in the vicinity of a person being measured.

### DETAILED DESCRIPTION OF THE PREFERRED EMBODIMENTS

The detailed description of the present invention is presented largely in terms of procedures, steps, logic blocks, processing, or other symbolic representations that directly or indirectly resemble the operations of data processing devices. These descriptions and representations are typically used by those skilled in the art to most effectively convey the substance of their work to others skilled in the art. Numerous specific details are set forth in order to provide a thorough understanding of the present invention. However, it will become obvious to those skilled in the art that the present invention may be practiced without these specific details. In other instances, well known methods, procedures, components, and circuitry have not been described in detail to avoid unnecessarily obscuring aspects of the present invention.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments mutually exclusive of other embodiments.

The present invention pertains to a system, a method, a platform and an application each of which is invented, uniquely designed, implemented or configured to cause a server device to receive sensor data captured from a subscriber or a user and detect his/her emotion. As used herein, any pronoun references to gender (e.g., he, him, she, her, etc.) are meant to be gender-neutral. Unless otherwise explicitly stated, the use of the pronoun “he”, “his” or “him” hereinafter is only for administrative clarity and convenience. Additionally, any use of the singular or to the plural shall also be construed to refer to the plural or to the singular, respectively, as warranted by the context.

One of the benefits, advantages and objectives in one embodiment of the present invention is to detect an emotional state of minds in a person based on collected biological data, at least some of which are collected directly from the person, where an emotion includes at least six characters: anger, disgust, fear, happiness, sadness and surprise. As will be described below, these characters can be presented in an index or a numeral with a range for the general public to understand what it means in the measured emotion. Further, different from medical tests conducted in a hospital, the biological data is largely collected over time by at least one wearable device carried by a user, where the user is not restricted to a particular location, a particular motion or a particular state.

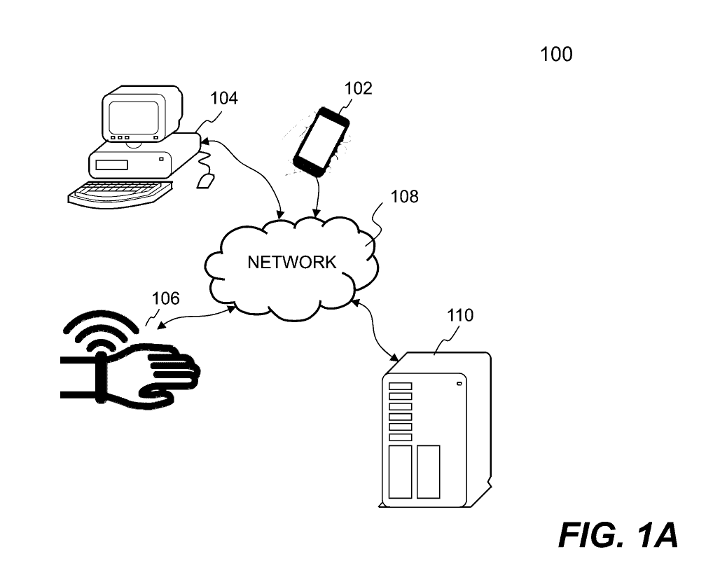

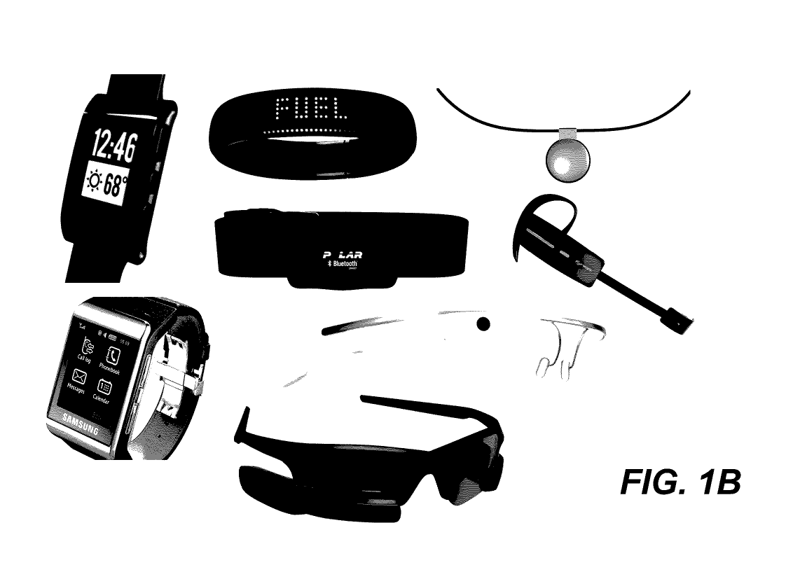

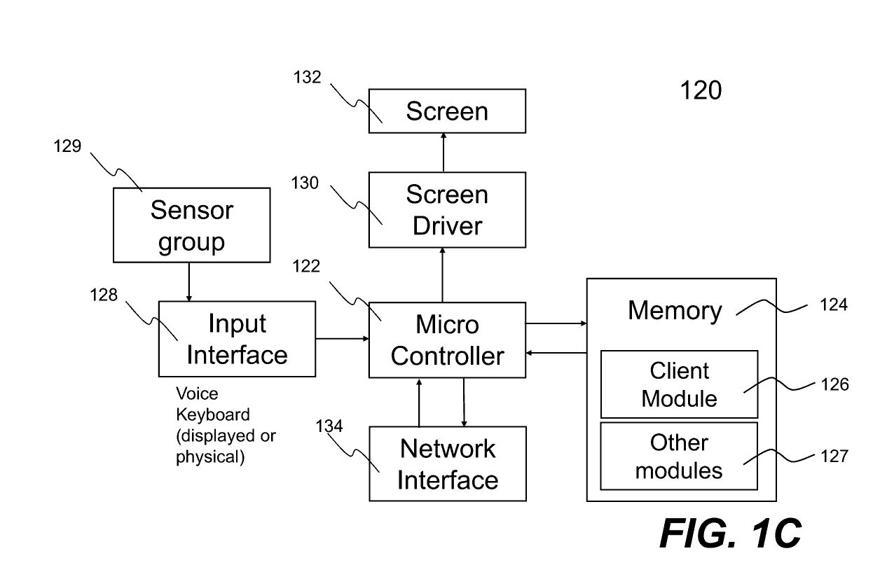

Referring now to the drawings, in which like numerals refer to like parts throughout the several views. FIG. 1A shows a basic system configuration 100 in which the present invention may be practiced in accordance with one embodiment thereof. FIG. 1A shows that there are three representative computing devices 102. 104 and 106, where the device 102 or 106 is meant to be a mobile device (e.g., a wearable device, a smart phone, a tablet or a laptop) while the device 104 is meant to represent a stationary device (e.g., a desktop computer). Each of the devices 102, 104 and 106 is loaded with a program, an application or a client module. In particular, Each of the devices 102, 104 and 106 is associated with a user, some of the devices 102, 104 and 106 are preferably to have a man-machine interface (e.g., a touch-screen display as most of the mobile devices do). Although other man-machine interfaces are possible, a touch-screen display provides the convenience for a user to interact with the device and to control when to allow the device to collect or transport biological data to a designated server 110.

FIG. 1B shows some of the commercially available wearable devices that may be used to collect one or more types of the biological data. Wearable devices such as activity trackers are a good example of the Internet of Things as they are part of the network of physical objects or “things” embedded with electronics, software, sensors and connectivity to enable objects to exchange data with a manufacturer, an operator and/or other connected devices, without requiring human intervention. One of more of the exemplary wearable devices shown in FIG. 1B may be used in FIG. 1A. Although it is possible to integrate many functions into a wearable device, it is well known that many of the wearable devices work in conjunction with a smartphone. For example, an Apple watch relies on a wirelessly connected iPhone (e.g., iPhone 5 or above) to perform many of its default functions (e.g., email and texting). Unless explicitly stated, a wearable device as described herein is assumed to work independently, capable of collecting biological data and transporting the data to a designated server (e.g., the server 110 of FIG. 1A) with or without a separate device (i.e., a smartphone or a desktop via a wireless link). Accordingly, a client device and a wearable device are interchangeably used herein.

According to one embodiment, the wearable device 106 includes a plurality of sensors. Examples of the sensors may include inertial measurement units (IMUs—including accelerometers, gyroscopes, magnetometer and barometers), optical sensors (including optical heart rate monitoring, PPG and cameras), electrodes, chemical sensors, flexible stretch/pressure/impact sensors, temperature sensors, microphones, and other emerging sensors. The details of the sensors are omitted herein to avoid obscuring aspects of the present invention. It is understood to those skilled in the art that various biological data, depending where the wearable device is worn on a body, can be captured.

According to one embodiment, a server device 110 is provided to administrate and execute some or all of an emotion evaluation process. In general, the server device 110 is provided to service a plurality of users and thus maintain a plurality of accounts, each corresponding to a subscriber, a member, or a user who has authorized to release the captured biological data to the server device 110. For simplicity, server device and server are interchangeably used hereinafter, so are client and client device. Accordingly, FIG. 1A shows a server executing a server module is in data communication with a plurality of clients, each of the clients executing a client module, where the server module or the client module implements one or more embodiments of the present invention.

Referring now to FIG. 1C, it illustrates an internal functional block diagram of an exemplary wearable device or client 120 that may be used as a client in FIG. 1A. The client 120 includes a microprocessor or microcontroller 122, a memory space 124 (e.g., RAM or flash memory) in which there is a client module 126, an input interface, a screen driver 130 to drive a display screen 132 and a network interface 134. The client module 126 may be implemented as an application implementing one embodiment of the present invention, and downloadable over a network from a library (e.g., Apple Store) or a designated server.

The input interface 128 includes one or more input mechanisms. A user may use an input mechanism to interact with the client 120 by entering a command to the microcontroller 122. Examples of the input mechanisms include a microphone or mic to receive an audio command and a keyboard (e.g., a displayed soft keyboard) to receive a click or text command. Another example of an input mechanism is a camera provided to capture a photo or video, where the data for the photo or video is stored in the device for immediate or subsequent use with other module(s) or application(s) 127. As part of the input interface 128, a plurality of sensors 129 are provided to capture a number of biological data from a user. Depending on implementation, some the sensors are integrated with the client device 120 and others may be peripheral or auxiliary to the client device 120. In addition, the mic and the camera are part of the sensors to capture an audio from the user and an image of a certain body part of the user. As will be explained further herein, there are two wearable devices worn by a user, each being equipped with different sensors and worn on a different part of the body, thus collecting different sets of biological data. The biological data is then transported via a single network interface or two different network interfaces to a server that is caused to proceed to determine the motion collectively on the sets of biological data and other data retrieved by the server.

The driver 130, coupled to the microcontroller 122, is provided to take instructions therefrom to drive the display screen 132. In one embodiment, the driver 130 is caused to drive the display screen 132 to display an image or images or play back a video. In the context of the present invention, the display screen 132 may display a message or an offer related to the detected emotion of the user. For example, when the emotion is detected “frustration” in conjunction with a long-delayed traffic jam, the display screen 132 is caused to display an offer to the user, where the offer may be related to an alternative route, a light music, a listening book or a recommended conversation with a loved one. The network interface 134 is provided to allow the device 120 to communicate with other devices via a designated medium (e.g., a data network such as HTTP or bluetooth link).

According to one implementation, the client module 126 is loaded in the memory 124 and executed by the controller 122 to capture some or all of the designated biological data from certain parts of the body. As will be further described below, the biological data is transported to the server 110 whenever a data link (e.g., WiFi) becomes available. Depending on how the data is captured and/or used, the client module 126 reports back to a server (e.g., the server 110 of FIG. 1A), where a profile of the user is updated. In one embodiment, the user is shown a message related to his confirmed emotion, where the message may be an advertisement (e.g., hypertension treatment when the blood pressure is detected consistently high for a period) or a service being offered (e.g., a doctor is linked to assess a condition beyond normal).

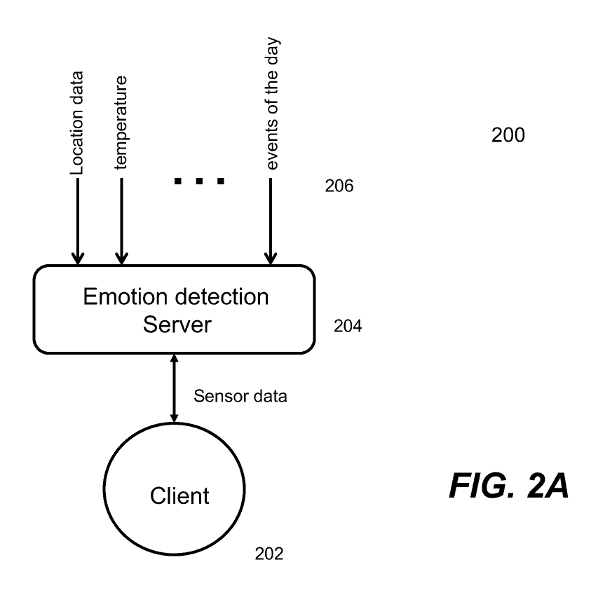

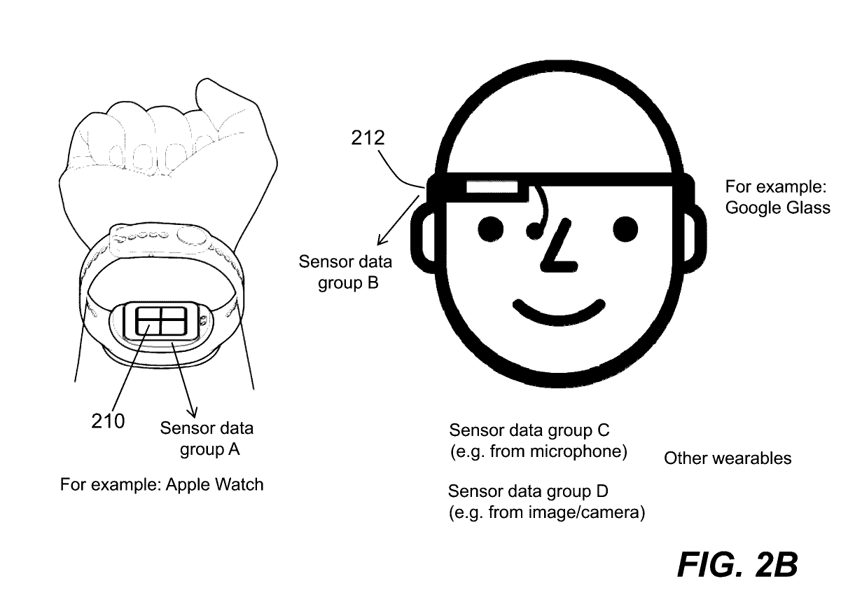

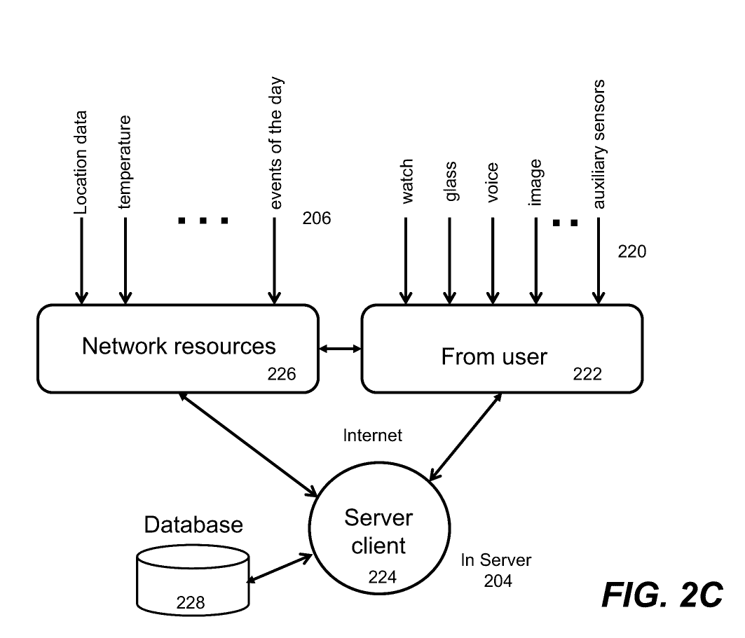

Referring now to FIG. 2A, it shows a logic relationship 200 between a client 202 and a server 204. The client 202 represents one of many clients that are intended to communicate with the server 204. In operation, the server 204 may be scheduled to request a client module in each of the clients respectively with the subscribing clients to send a set of collected biological data. Users of the clients are assumed to have signed up with the server 204 and authorized the data to be sent securely to the server 204. The client 202 is caused to execute a client module that drives a plurality of sensors provided to capture biological data from one or different parts of the body. In one embodiment, the client module is an application running in a smartphone and drives the equipped sensors to collect predefined data.

Once a set of data from a user is received in the server 204, according to one embodiment, the server 204 executes a server module that is invented, uniquely designed, implemented and configured to determine an emotional status of the user in accordance with real-time data collected from other sources available on the network. For example, besides the biological data from the client, various situations at or in the vicinity of the location where the user is located, weather conditions of the location, or various related events of the day near the location may be used in determining the emotion of the user. Further the profile of user may also be used or at least referenced in determining the emotion of the user. For example, the emotion of the user may be detected that the user seems to be in a mood of dismay. The emotion determination can be concluded that the user may be in anxiety when the profile indicates that the user is interested in stock investment and there happens to be a sudden drop of over 500 points in Dow Jones Industrial Average (DOW).

Emotion is a natural instinctive state of mind deriving from one's circumstances, mood, or relationships with others. Although scientific discourse has drifted to other meanings and there is no consensus on a definition, emotion is often intertwined with mood, temperament, personality, disposition and motivation. To assist a user in general to understand his state of feeling that may result in physical and psychological changes, a type of expression is used to indicate to the user that his state of feeling may influence his logical thinking, wellbeing or his behavior emotional status. Depending on implementation, the determination of emotion is represented in different expressions. Besides word expressions such as sad, anger, dismay, joy, happy or excited, the emotion can be expressed with a ranking, an index or a level with a range. For example, a quantitative (numerical) indication in a range of 1˜10 is used to indicate that the emotion index being 1 is in the saddest mood (e.g., very sad) and the emotion numeral being 10 is in the happiest mood (e.g., excited). Logically, average persons with an emotion index of 5 or 6 would be considered neutral while the emotion index falling between 6 and 9 would be desirable. As will be detailed below, the emotion index can be used to trigger many useful services or applications in the context of the present invention.

In general, positive emotions tend to broaden an individual's momentary thought-action repertoire. Users are able to analyze and react appropriately or make better decision in perceiving a certain situation. It can help to loosen the hold on negative emotions gained on an individual's mind and body. By then, it will recover the speed of cardiovascular better compared to negative emotions. Though not accurate, the numerical presentation of an emotion described herein provides a relative indication of a mood feeling a person is having and can be a reference value for many corresponding services or goods to follow.

Referring now to FIG. 2B, it shows two wearable devices, a watch (e.g., Apple Watch) and a pair of glasses (e.g., Google glass) that may be used to capture some of the biological data. It is well known that an Apple Watch is equipped with four sensors 210 to measure the pulse of its wearer. The sensors 210 include infrared and visible-light LEDS in addition to photosensors, which all work together to detect a heart rate. Given the limited number of sensors that are nearly all focused on the wrist part of the wearer, the biological data being captured as Sensor Data Group A may not be sufficient to determine the emotion of the wearer. Google glass includes well over 10 different sensors 212 and can generate Sensor Data Group B. Apple Watch and Google glass are located on different parts of a body and good to capture similar or different biological data from two different locations. For example, a body temperature may be sampled from the arm (i.e., by a wrist device) and the head (i.e., by a pair of glasses). The correlated data, most likely different on the different parts of the body, may be used in determining the emotion of the wearer.

According to another embodiment, an auxiliary device with at least one or more sensors may be carried by a user. An example of such sensors that may be integrated in a wearable device or a separate device includes a biometric skin sensor from Vital Connect located at 900 East Hamilton Ave, Suite 500 Campbell, Calif. 95008. Those skilled in the art may appreciate that more sensors may be used across a body as long as they are integrated conveniently. In addition to the sensor data groups from at least two different locations of a body, a voice and/or a facial image may also be collected as additional sensor data group(s). At a certain point, the data together with other inputs from the user, all referred to as biological data, is transferred to a designated server.

FIG. 2C illustrates that the biological data 220 captured from a user is transported to a server 204 with other external data. Depending on implementation, a secured communication channel may be established between the client 202 and the server 204 to allow the biological data 220 to be uploaded from the client 202 to the server 204. In operation, the client module in the client device 202 is caused to contact the server module in the server 204. After a few data exchanges including verification of the user, a secured session is established to allow the biological data 220 to be uploaded to the server 204. According to one embodiment, the server 204 is caused to calculate an emotion measurement from the biological data 220 with or without historical biological data of the same user. According to another embodiment, network resources 226 are selectively retrieved by the server module 224 to better understand the biological data 220. As indicated above, some of the biological data would only make sense in conjunction with the external, ambient or surrounding conditions at the time the data was captured. For example, it is reported from one network resource that there is a thunderstorm going on in the area of the user, the emotion index would have to be re-adjusted or re-computed when it is detected from the received GPS data that the user is driving on road that is being hit hard by the thunderstorm (e.g., resulting in a lower emotion index value).

As will be further detailed below, according to one embodiment, the server client 224 is caused to retrieve historical data of the user from a database 228. The historical data is defined as any data captured from the user or provided by the user prior to the moment that the emotion of the user is determined. The historical data may include the biological data received in the past, some or all of the retrieved network resources and references to the profile that may be periodically updated in connection to some events that may have happened to the user.

As an example of using the historical data, a heart rate in the biological data 220 from a user is well over or beyond an averaged value of the user and is made to contribute a little in determining the emotion when it is detected that the user is in the middle of exercising or often involved in a sport activity around the time in the past. Similarly, a higher body temperature in the biological data 220 would not cause an alert in determining the emotion when it was already in record that the user has been experiencing a fever due to his recent exposure to flu.

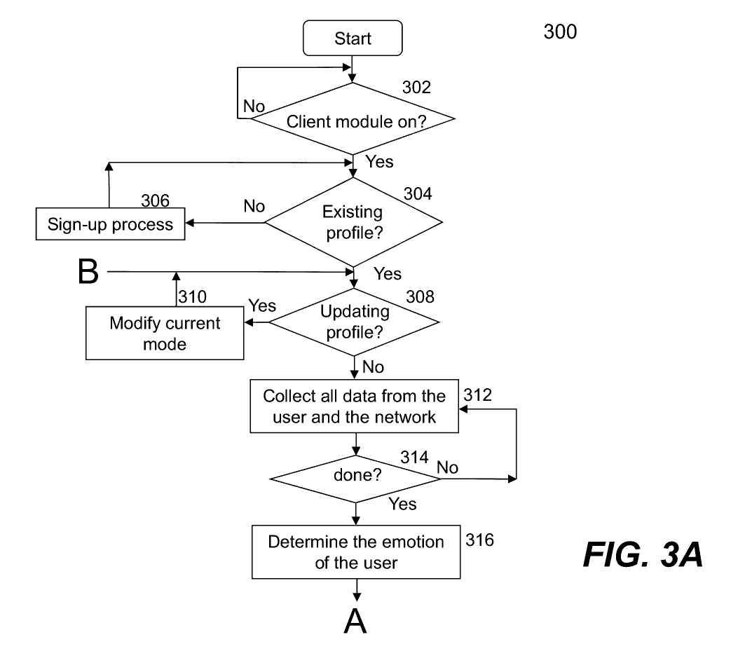

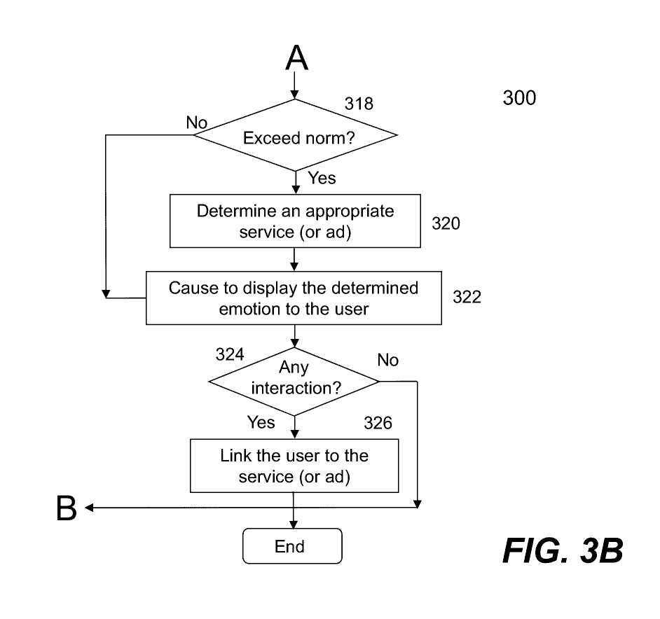

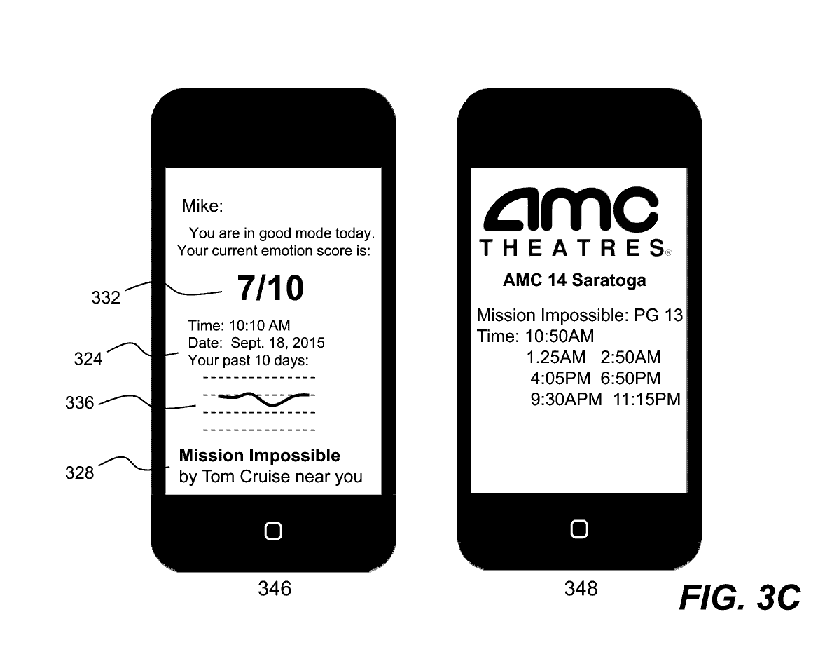

Referring now to FIG. 3A and FIG. 3B, it shows a flowchart or process 300 of determining an emotion for a user from the biological data captured directly from the user and other available data from the Internet. As will be appreciated by those skilled in the art that the process 300 is not something a general computer is capable of performing by itself. A general computer must be specifically programmed or installed with a specifically designed module according to one embodiment of the present invention, resulting in significantly more than what a general computer is designed to do. As will be further demonstrated, the process 300 undertaken between two computing devices (e.g., a server and a client) is not a collection of human activities as it is practically impossible by any measure for some of the procedures to be performed by or to involve the intervention of human beings. With the execution of a client module or a server module implementing one embodiment of the present invention, the two computing devices (e.g., a smartphone and a server computer) are caused to perform beyond what they are originally capable of or meant to do. The process 300 may be understood in conjunction with the preceding drawings and may be implemented in software or a combination of software and hardware.

It is assumed that a user is using a client (e.g., a smartphone or a computer) that has been installed with a client module (e.g., the module 126 of FIG. 1C). The module is activated manually or automatically upon an event. At 302, the process 300 can only proceed when the module is running. Depending on situation, the user may manually activate the client module by clicking on an icon or link representing the client module or the client module is automatically activated by an application, a webpage being visited, a stored cookie or at a specific time.

The process 300 proceeds to 304 where a profile of the user is examined. If it is the first time the user uses the process 300 (e.g., the emotion determination service), the user will be directed to 306, where the user is requested to complete a sign-up process. Depending on implementation, the sign-up process may require some or all of the following: real name of the user, residential address, email address, his profession or hobbies, his general health parameters (weight, height, blood pressure and etc.), what kind of outdoor or indoor activities he is interested or sometimes his financial status. In addition, there may be one or more questions of what the user is planning to do immediately, in a week or a month or so if there is an opportunity (e.g., a vacation, to purchase a house or to sell/but stock of a company). The question(s) may be supplemented with questions of any preferred brand, model, size, color, quantity, or price range, and etc. In one embodiment, the user is asked if a relevant ad can be served before, during or after his emotion is evaluated. If the user has already established an account with the server (e.g., on the server 110 of FIG. 1A), the process 300 goes to 308 to check if the user needs to update his account and/or profile.

The process 308 may not appear every time but assist the user to update his profile when there is a need. Sometimes, the user has purchased something somewhere else while the profile still indicates that the user is planning to purchase the item, in which case the profile is preferably updated. Should the user choose to modify his profile, the process 300 goes to 310, where the user may be asked for his current mode (e.g., a level of his comfort with something). Once there is no more update to the profile at 308, the process 300 goes to 312 to start what is referred to as a biological data collection phase.

It should be noted that various data is not necessarily collected simultaneously. In operation, many are collected over a period of time provided a client module is running in a wearable device. For example, body temperature may be captured overtime and be cached in the device. The temperature data, most likely varying over time, may be averaged or filtered and a representative thereof is sent to the server to represent a body temperature at the time of collection. Similarly, a heart rate is collected periodically or at predetermined times. When the heart rate is called to be collected in the server, the data representing the heart rates over a period of time may be processed (e.g., averaged or filtered) and a representative thereof is sent to the server to represent the heart rate of a user at the time of collection. It shall be understood to those skilled in the art that the processing of the data may be carried out locally or in the server with more sophisticated approaches, in either case, all collected or required data in the client may be transported to the server in a batch.

Meanwhile at 312, the server module is invented, uniquely designed, implemented or configured to cause the server to retrieve all relevant data from predefined network resources. Depending on the profile of the user, a service length the user has signed up with the server, and a service level, a set of predefined network resources are defined in accordance with a set of data including the collected biological data, his profile, his current location, time and date. In one example, a weather website (e.g., www.weather.com) is visited and weather data for the location where the user is currently located or nearby is obtained. A traffic reporting website (e.g., maps.google.com) is visited and traffic data for a location where the user is currently located or nearby is obtained when it is noticed that the user is on road. A stock market website may also be visited and stock data for a set of symbols (e.g., NASDAQ index) is obtained when it is noticed that the user is an active trade in the stock markets.

At 314, the process 300 ensures that all pre-determined data is obtained, retrieved or collected. The process 300 is now moving to 316 where the emotion of the user is determined or calculated. Depending on implementation, various algorithms or schemes may be applied to the collected data to determine the emotional status of the user. According to one embodiment, the neural network or machine learning is used. An artificial neural network (ANN) is an information processing paradigm that is inspired by the way biological nervous systems, such as the brain, process information. The key element of this paradigm is the novel structure of the information processing system. It is composed of a large number of highly interconnected processing elements (neurones) working in unison to solve specific problems. ANNs, like people, learn by example. An ANN is configured for a specific application, such as pattern recognition or data classification, through a learning process. To avoid obscuring aspects of the present invention, details of the neural network are omitted. Those skilled in the art know there are publicly available rich sources describing the neural network in detail.

At 318, the result from the determination of 316 is examined to see if the result is out of a normal range. When the process 300 is noticed that the result is exceeding a predefined normal range, the process 300 goes to 320 that is configured to determine an appropriate service. At 322, a display is caused to show such a service including an advertisement. Depending on the display in a smartphone or a wearable device, the suggested service may be presented as a link, in a text or a multimedia display. For example, the emotion derived from all the collected data indicates that the user is nearly upset or angry, a suggestion of a light music (via a link) is provided to the user. In another service, a medical/health provider is suggested when it is noticed that the blood pressure of the user is consistently higher than the average range in the same aged group for a period of time.

Returning to 318, when the process 300 notices that the emotion or any of the collected biological data is within a predetermined range, the process 300 goes to 322 to display the derived emotion to the user, possibly along with one or more suggested services. FIG. 3C shows an example of the display 346, where there are a numerical expression 332 of the measured emotion when the emotion is measured, and a curve 336 to show a set of past measurements so that the user can see how his mode has changed over the period. In addition, the display 346 shows an advertisement 338 that is determined that the user is likely to activate it given his emotion at the moment. The display 348 shows the detail of the advertisement after the user has activated with the advertisement 338 in the display 346.

Returning back to FIG. 3B, at 324, the process 300 monitors whether the user interacts with any of the suggested service (including a displayed advertisement). The mentoring process is generally performed by the client module. When the user interacts with one of the displayed suggested service or advertisement, the client module records when and how the user has interacted with the displayed suggested service or advertisement. The action may be used to update the profile of the user so that a more appropriate service or advertisement may be delivered to the user next time when there is an opportunity. At 326, after the user activates with the displayed service, the process 300 ends and the user is brought to a website linked by the displayed suggested service.

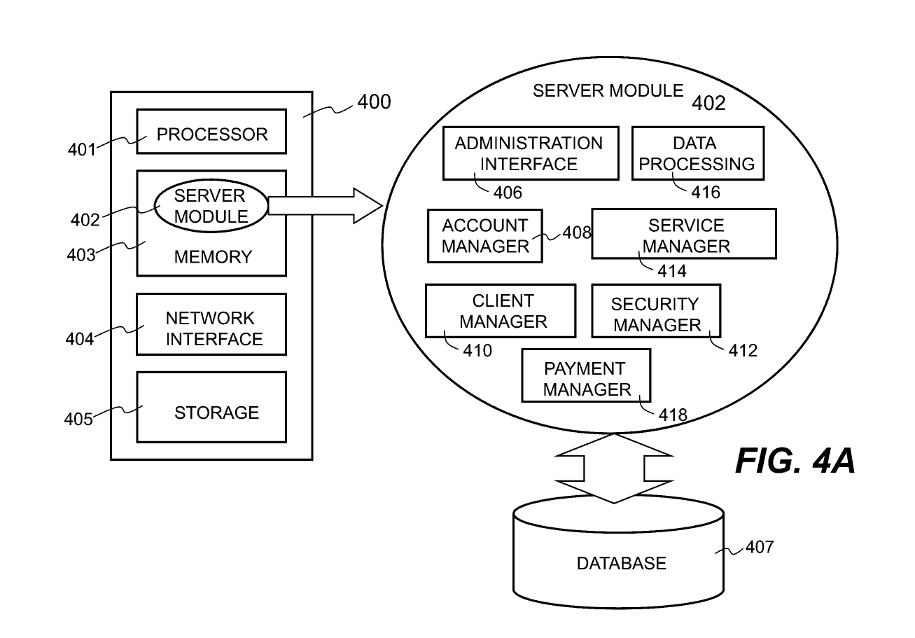

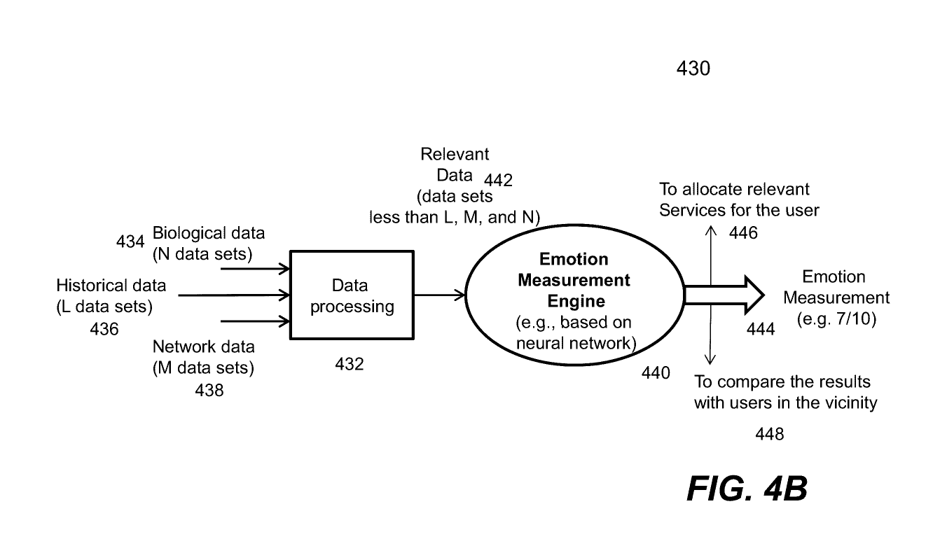

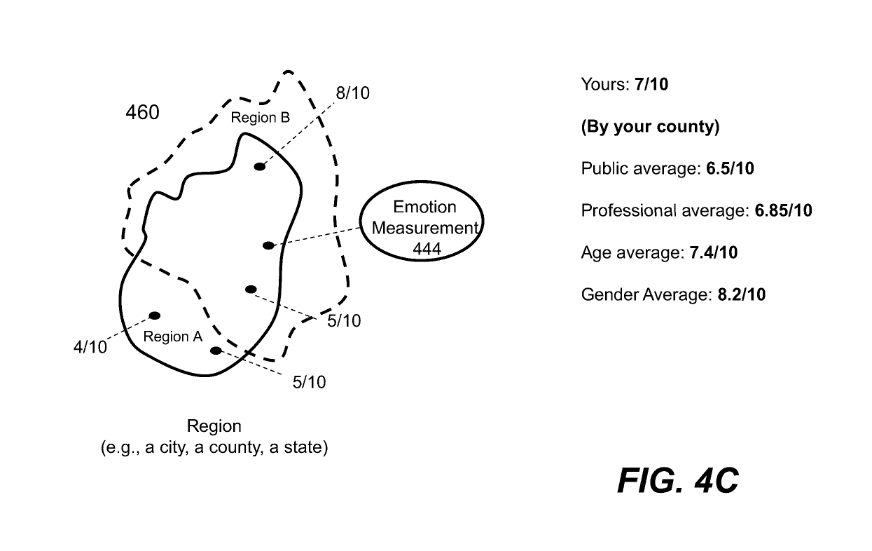

Referring now to FIG. 4A, there is shown a functional block diagram of a server 400 in which a server module 402 resides in a memory space 403 and is executed by one or more processors 401. The server 400 is a representation of many similar servers operated by a service provider and may be used in FIG. 1A to determine an emotion state for each of subscribers or users, make an arrangement between a service provider (e.g., an advertiser) and each of the users, and settlements of payments or points towards the use of an advertisement.

Depending on implementation, this server 400 may be a single server or a cluster of two or more servers. One embodiment of the present invention is implemented as cloud computing in which there are multiple computers or servers deployed to serve as many businesses or individuals as practically possible. For illustration purpose, a single server 400 is shown in FIG. 4A. Further, the server 400 includes a network interface 404 to facilitate the communication between the server 400 and other devices on a network, and a storage space 405. In one embodiment, the server module 402 is an executable version of one embodiment of the present invention and delivers, when executed, some or all of the features/results contemplated in the present invention. It should be noted that a general computing device is not able to perform or deliver what the server 400 is equipped to do without the installation of or access to the server module 402.

According to one embodiment, the server module 402 comprises an administration interface 406, an account manager 408, a client (advertiser) manager 410, a security manager 412, an service manager 414, a data processing module 416 and a payment manager 418.

### Administration Interface 406:

As the name suggests, the administration interface 406 facilitates a system administrator to access various components in the server module 402 and set up various parameters of the components. In one embodiment, a service provider uses the administration interface 406 to determine a subscription fee (e.g., a certain amount to free per month for an account) for each of its subscribers or a service level depending on how much a subscription fee is paid. For example, a subscriber paying a fee gets access to a record for all past measurements, share one or more results with his contacts (knowingly or anonymously), or compare his own with some of his contacts or a group of similar users. A user paying nothing is limited to his current emotion measurement and may be served some advertisements when viewing his result. In another embodiment, the administration interface 406 allows a service provider to manage all subscribing accounts for the advertisers and determine what and how much to charge for servicing the advertisers. In addition, advertisements in digital forms are received from the advertisers and kept in storage 405 or a database 407 via the administration interface 406.

### Account Manager 408:

The account manager 408 is provided to allow a user to automatically register himself with the server 400 for a service being offered by the server 400 or registered with a client module running on his mobile or wearable device(s), where the client module is designed to cause his mobile device to communicate with the server 400 via the interface 404. In one example, a user causes the client module to be executed for the first time on his device (e.g., iPhone or Apple Watch), the module is designed to request the user to enter certain information (e.g., username/password, a fingerprint, a true name and etc.) before allowing the user to create a profile, part of which can be periodically updated by the server 400 per data received related to the user. In one embodiment, a user is allowed to link his electronic wallet to his account. Whenever there is a payment request, the payment can be made directly from his electronic wallet. After the registration, a profile of the user is created and then transported to the server 400. In one embodiment, the account manager 408 is designed to augment the profile with a system-created portion so that any updates to the profile will be stored in the portion to better serve the user.

### Client Manager 410

The client manager 410 is provided to manage versions of client modules provided to the users. In one embodiment, besides keeping updates to the client module, there may be two versions of it, one for users who pay subscription fees, and the other one for non-paying users. Depending on implementation, the version for the paying users may include more functions to provide the users with more customized services opted by the user while the version for the non-paying users may include some services that require some actions from the user to benefit the provider one way or the other. In one embodiment, these two versions of the client module may be implemented as a single module or two separate modules. In the context of the present invention, the client manager 410 controls when to switch from one version to another in accordance with a set of parameters about a user. In operation, the client manager 410 is notified which version or release a registered user is using. Further, the client manager 410 provides necessary information when it comes to deliver a type of service or advertisement to a user.

### Security Manage 412

This module is configured to provide data security when needed. The stored data for each of the subscribing businesses or registered users may be encrypted, thus only an authorized user may access the secured data. For example, all personal information of the users, especially the accounts set up by the users to obtain their emotion measurements are stored securely. In one embodiment, the security manage 412 is configured to initiate a secure communication session with a client device when the biological data of the user is transported to the server. In addition, the profile and any preferences provided by the user are also secured by the security manager 412.

### Service Manager 414

The service manager 414 is a tool provided to allocate one or more services (e.g., advertisements of certain goods and services) for a user in accordance with his provided or updated profile, where the services are chosen based on certain criteria set by the service provider or/and the user. Depending on implementation, the criteria may be based on a profile provided by the user or a profile retrieved from a social network, where the user allows an access to his profile on the social network and shares his interests with others there. In operation, the Service manager 414 is designed to allocate advertisements for each of the users based on their measured emotion data to maximize the delivery and usefulness of the respectively allocated advertisements.

### Data Processing 416:

This module is configured to perform analytic operations to determine what network resources shall be used and what portion of the biological data to be used in determining the emotion of the user. Given the information provided by a user and/or collected about the user from the historical data, the data processing module 416 determines a set of data deemed the mostly appropriate to measure the emotion of the user at the time the emotion is set to be measured. FIG. 4B shows a functional block diagram 430 according to one embodiment. A data processing unit 432 is designed to receive some or all of the biological data sets 434, historical data sets 436 and network data sets 438. The biological data sets 434 include the latest captured biological data set from the user and perhaps some or all of the previously captured biological data sets from the user. The historical data sets 436 include past measurements or special notes to some of the measurements. The network data sets 438 include current and previous relevant data from the Internet. The data processing unit 432 is designed to filter out some of the data sets that may introduce errors to the current measurements. According to one embodiment, the data processing unit 432 is configured to take out some extremes, namely those data sets are so far away from the norm. As a result, the outputs 442 from the data processing unit 432 have a less number of data sets than the input receives. The outputs 442 from the data processing unit 432 are then provided to the emotion measurement engine 440 to determine what emotion the user may have now.

### Payment Manager 418:

As the name suggests, this module is designed to settle the payment with a user should there be a need for payment from the user or from the service provider. In operation, this module works with the account manager 408 to ensure a payment is securely settled with an electronic wallet designated by the user. As described above, when viewing an ad, the user may click it though, result in a transaction from it. In one embodiment, the payment manager 418 settles the payment towards the completion of the transaction.

Referring now back to FIG. 4B, the measured emotion 444 from the engine 440 is converted to an index expression that can be compared to a predefined threshold. In one embodiment, the result 444 is used to determine what service is appropriate to the user given the measured emotion thereof. FIG. 4C shows a diagram of comparing the measurement 444 in the vicinity of the user. A geographic region may be manually defined by the user to see a comparison of his own emotion measurement with others in specific groups, such as a group defined by general public (regardless of the gender, age, profession or others). It is described above that the server 400 is designed and configured to maintain a plurality of users. Over the time, each of the accounts would have accumulated a series of emotion measurements. According to one embodiment, these measurements can be used anonymously for different purposes. Since each account includes some of the basic information, such as age, residential location, gender, profession. Thus the accounts can be sorted and the measurements thereof can be used, for example, to show an averaged measurement in a group in a region by gender, age, profession or others.

In one embodiment, the user is allowed to define on his smartphone a region to compare his measured emotion with others in the region by specifying a common character in the group (e.g., gender, age, profession). Depending on need, the user may define one or more cities, counties and states as a region and may further define what type of groups to be compared with. FIG. 4C shows that the group may be based on a specific type, resulting in the averaged measurement from the group in the region. As indicated above, the emotional status of a human being is subjective, so is the calculated emotion index. Given the option to see what others are having, a user shall appreciate the emotion index being displayed on the display screen of his mobile device.

The invention is preferably implemented in software, but can also be implemented in hardware or a combination of hardware and software. The invention can also be embodied as computer readable code on a computer readable medium. The computer readable medium is any data storage device that can store data which can thereafter be read by a computer system. Examples of the computer readable medium include read-only memory, random-access memory, CD-ROMs, DVDs, magnetic tape, optical data storage devices, and carrier waves. The computer readable medium can also be distributed over network-coupled computer systems so that the computer readable code is stored and executed in a distributed fashion.

The present invention has been described in sufficient details with a certain degree of particularity. It is understood to those skilled in the art that the present disclosure of embodiments has been made by way of examples only and that numerous changes in the arrangement and combination of parts may be resorted without departing from the spirit and scope of the invention as claimed. Accordingly, the scope of the present invention is defined by the appended claims rather than the foregoing description of embodiments.

I claim:

1. A method for measuring an emotion, the method comprising:
retrieving a profile of a user;
sending a request by a server device to a client device to capture some or all of predefined biological data from the user, wherein at least a part of the client device is wearable and includes a plurality of sensors generating different sensing data;
receiving the biological data from the client device;
feeding the biological data to a data processing unit together with other data;
providing processed data to an emotion measurement engine configured to derive the emotion from the processed data; and
causing the client device to display the derived emotion to the user.

2. The method as recited in claim 1, wherein the client device communicates wirelessly with the server, the client device is caused to execute a client module configured to collect the sensing data from the sensors.

3. The method as recited in claim 2, wherein at least one of the sensors is not close to proximity of other sensors.

4. The method as recited in claim 3, wherein the biological data includes a voice from the user.

5. The method as recited in claim 4, wherein the biological data includes an image of the user.

6. The method as recited in claim 1, further comprising:
obtaining the other data from a number of predefined network resources, wherein the other data pertains to a location of the user, a condition of the location or any event related to the user or the location.

7. The method as recited in claim 6, further comprising retrieving historical data about the user.

8. The method as recited in claim 7, wherein said feeding the biological data to a data processing unit together with other data comprises:
removing some of the biological data and the other data so that remaining biological data and other data are relevant to the user at the time of collecting the biological data.

9. The method as recited in claim 1, wherein said causing the client device to display the derived emotion to the user comprising:
providing one or more service links to the user in connection with the derived emotion; and
monitoring if the user has interacted with any of the links.

10. The method as recited in claim 9, wherein the profile is updated whenever any of the links is activated by the user.

11. A mobile device for measuring an emotion, the mobile device being carried by a user and comprising:
a plurality of sensors
a processor;
a wireless interface to allow the mobile device to communicate with a server device wirelessly over a data network;
a memory space, coupled to the process, provided to store a client module, wherein the client module is executed by the process to cause the mobile device to perform operations of:
collecting some or all of predefined biological data of the user from the sensors in response to a request from the server device to capture the biological data;
transporting the biological data to the server device;
receiving a derived emotion measurement from the server device, wherein the server device is configured to derive the emotion of the user from the biological data and other data; and
displaying the derived emotion to the user.

12. The mobile device as recited in claim 11, wherein at least one of the sensors is not close to proximity of other sensors.

13. The mobile device as recited in claim 12, wherein the biological data includes a voice from the user.

14. The mobile device as recited in claim 13, wherein the biological data includes an image of the user.

15. The mobile device as recited in claim 11, wherein the operations further comprises:
providing one or more service links to the user in connection with the derived emotion; and
monitoring if the user has interacted with any of the links.

16. The method as recited in claim 15, wherein the operations further comprises causing the profile to be updated whenever any of the links is activated by the user.

17. A method for measuring an emotion, the method comprising:
retrieving a profile of a user;
sending a request by a server device to a client device to cause at least two wearable devices to capture some or all of predefined biological data from the user, wherein each of the wearable devices includes a plurality of sensors generating different sensing data;
receiving the biological data from the client device;
feeding the biological data to a data processing unit together with other data;
providing processed data to an emotion measurement engine configured to derive the emotion from the processed data; and
causing the client device to display the derived emotion to the user.

18. The method as recited in claim 17, further comprising:
obtaining the other data from a number of predefined network resources, wherein the other data pertains to a location of the user, a condition of the location or any event related to the user or the location.

19. The method as recited in claim 18, further comprising retrieving historical data about the user.

20. The method as recited in claim 17, wherein said feeding the biological data to a data processing unit together with other data comprises:
removing some of the biological data and the other data so that remaining biological data and other data are relevant to the user at the time of collecting the biological data.
